SEQUENCE LISTING (33 sequences)

SEQ ID NO: 1 - DNA, 3711 bp, Saccharomyces cerevisiae
  source 1..3711: mol_type = genomic DNA
atgcattttagaagaacgatgagtagagtgcccacattggcatctcttgaaatacgatataaaaaatctttcggccataaatttcgtgattttattgctctatgtggtcactattttgctccagttaaaaaatatatcttccccagttttatcgcggttcactacttctacacgatatccctgacattaataacttcaatcctgctatatcccattaagaataccagatacattgatacattgtttttagcagcgggcgcagttacacaaggtggcttaaatactgtggatatcaataatctaagcttataccaacaaattgttctgtatatcgtatgctgcatatcaacaccaattgcagttcatagttgcttggcatttgtacggctttactggtttgagcgctacttcgatggtattagagactcttctagacgaaattttaagatgagaagaacgaaaacaatcttagaaagggaactaacagcaagaaccatgaccaagaatagaacaggtacccaaagaacgtcttatcctaggaaacaagctaaaacagatgatttccaagaaaaattgttcagcggagaaatggttaatagagatgagcaggactcagttcacagcgaccagaactctcatgacattagtagggacagcagcaataataatatgaatcacaatggtagcagtgtcagtttagatgatttcgttaaggaagacgaaacggatgacaacggagaatatcaggagaacaactcctactcgacggtaggtagttcgtctaacacagttgcagacgaaagtttaaatcagaagcccaagccaagcagtcttcggtttgatgagccacacagcaaacaaagacccgcaagagttccctcagagaaatttgcaaaaagaaggggttcaagagatattagcccagccgatatgtatcgatccattatgatgctacaaggtaagcatgaagcaactgctgaagatgaaggtccccctttagtcatcgggtcccctgcggatggcacaagatataaaagtaatgtcaataagctaaagaaggccaccggcataaatggtagcacaatcaagattcgagataagggaaatgaaagtaacactgatcaaaattccgtgtcaagtgaagcaaacagtatggcgagcgtttcggacgaaagctcgttacccacaaattttggtaacaaagtaccttcattaagaacaaatactcatagatcaaattcgggcccgatagccattactgataacggagaaacagacaaaaagcatgggccatcaattcaattcgatataactaaacctcctagaaaaatttcaaaaagagtttcaaccttcgatgatttgaacccaaaatcttccgttctttatcgaaaaaaagcatcgaagaagtacctcatgaaacattttcctaaagcgcggcgaatacggcaacaaattaagagaaggctttctactggttcgattgagaaaaacagcagtaacaatgtttcagatagaaaacctattactgatatggatgatgatgatgatgatgacgatgacaacgacggcgataacaacgaagaatactttgctgacaacgaaagcggcgatgaaaatgaacgagtacagcagtctgaaccacattctgattcagaactcaaatcgcaccaacaacaccaagaaaaacaccaactgcagcagaacctgcaccgcatgtataaaaccaaatcatttgatgataatcgttcaagagctgttcctatggaacgttccaggaccatcgatatggctgaggctaaggatctaaatgagctcgcaaggacgcctgattttcaaaaaatggtctatcaaaattggaaagcccatcatagaaaaaaaccgaactttaggaagaggggatggaataacaagatatttgaacatggtccctatgcatctgacagcgatcgcaattatcctgataatagtaatactggaaacagtattcttcattacgcagaatctattttacatcatgatggctctcataaaaatggaagcgaagaagcctcttccgactctaatgagaatatctattccacgaatggaggaagcgaccacaatggtcttaacaactatcctacttacaacgacgatgaagaaggctattatggtttacatttcgataccgattatgacctagatcctcgtcatgatttatctaaaggcagtggtaaaacgtatctatcatggcaaccaactattggacgtaactcaaacttccttggattaacaagagcccagaaagatgaattaggtggtgtcgagtacagagcaatcaaacttttatgcaccatattggttgtctactacgttggatggcatattgttgcttttgttatgttagtaccttggattattttgaaaaagcattatagtgaagttgttagagatgatggtgtttcacctacatggtggggattttggacagcaatgagtgcatttaatgatttaggtttgacattaactccaaattcaatgatgtcgtttaacaaagctgtatacccattgatcgttatgatttggtttatcattatcggaaatacagggtttcccatccttcttagatgcatcatttggataatgtttaaaatttctcctgatttatcacagatgagagaaagtttaggttttctcttagaccatccacgtcgttgtttcaccttgctatttcctaaggcagctacatggtggctacttttaacgcttgcaggattgaatataactgattggattttatttattattctagattttgactcaacagttgtgaaatcattatcgaaaggctatagagtccttgtcggcctgtttcaatctgttagcacaagaactgctggattcagcgttgtcgatttaagtcaactgcatccttctatccaagtctcctatatgctaatgatgtatgtctccgtattaccattggccatctctattcgacggacaaatgtttacgaggagcaatctctaggaatatatggagatatggggggagaaccagaagatacggatactgaagacgatggtaacgatgaagatgacgacgaggaaaacgagagtcacgaaggtcaaagtagtcaaagaagtagttcaaacaacaacaacaataacaacaggaaaaagaaaaagaaaaagaaaactgaaaatccaaatgaaatatctacaaaatcctttatcggtgcccatttaaggaaacagctttcatttgacttgtggtttctatttttagggttatttatcatttgcatttgtgaaggggacaagataaaggacgtacaagaaccaaactttaatatatttgcaattctttttgaaattgttagcgcttacggtacagttgggctatcgctaggttatccggacaccaaccaatcgttttcaagacagcttactacattatctaagttggtgatcatagctatgctgatcagaggcaagaatagaggtctaccatactcactggatcgtgcaattatcttgcctagtgatagacttgaacattttgaccaccttgagggcatgaagttgaagagacaggctagaaccaatacagaagacccaatgacggaacatttcaagagaagtttcactgatgtgaaacatcgttggggagctcttaagcgtaagaccacacattcccgaaatcctaaaaggagcagcacaacgctctaa

SEQ ID NO: 2 - protein, 1236 aa, Saccharomyces cerevisiae
MHFRRTMSRVPTLASLEIRYKKSFGHKFRDFIALCGHYFAPVKKYIFPSFIAVHYFYTISLTLITSILLYPIKNTRYIDTLFLAAGAVTQGGLNTVDINNLSLYQQIVLYIVCCISTPIAVHSCLAFVRLYWFERYFDGIRDSSRRNFKMRRTKTILERELTARTMTKNRTGTQRTSYPRKQAKTDDFQEKLFSGEMVNRDEQDSVHSDQNSHDISRDSSNNNMNHNGSSVSLDDFVKEDETDDNGEYQENNSYSTVGSSSNTVADESLNQKPKPSSLRFDEPHSKQRPARVPSEKFAKRRGSRDISPADMYRSIMMLQGKHEATAEDEGPPLVIGSPADGTRYKSNVNKLKKATGINGSTIKIRDKGNESNTDQNSVSSEANSMASVSDESSLPTNFGNKVPSLRTNTHRSNSGPIAITDNGETDKKHGPSIQFDITKPPRKISKRVSTFDDLNPKSSVLYRKKASKKYLMKHFPKARRIRQQIKRRLSTGSIEKNSSNNVSDRKPITDMDDDDDDDDDNDGDNNEEYFADNESGDENERVQQSEPHSDSELKSHQQHQEKHQLQQNLHRMYKTKSFDDNRSRAVPMERSRTIDMAEAKDLNELARTPDFQKMVYQNWKAHHRKKPNFRKRGWNNKIFEHGPYASDSDRNYPDNSNTGNSILHYAESILHHDGSHKNGSEEASSDSNENIYSTNGGSDHNGLNNYPTYNDDEEGYYGLHFDTDYDLDPRHDLSKGSGKTYLSWQPTIGRNSNFLGLTRAQKDELGGVEYRAIKLLCTILVVYYVGWHIVAFVMLVPWIILKKHYSEVVRDDGVSPTWWGFWTAMSAFNDLGLTLTPNSMMSFNKAVYPLIVMIWFIIIGNTGFPILLRCIIWIMFKISPDLSQMRESLGFLLDHPRRCFTLLFPKAATWWLLLTLAGLNITDWILFIILDFDSTVVKSLSKGYRVLVGLFQSVSTRTAGFSVVDLSQLHPSIQVSYMLMMYVSVLPLAISIRRTNVYEEQSLGIYGDMGGEPEDTDTEDDGNDEDDDEENESHEGQSSQRSSSNNNNNNNRKKKKKKKTENPNEISTKSFIGAHLRKQLSFDLWFLFLGLFIICICEGDKIKDVQEPNFNIFAILFEIVSAYGTVGLSLGYPDTNQSFSRQLTTLSKLVIIAMLIRGKNRGLPYSLDRAIILPSDRLEHFDHLEGMKLKRQARTNTEDPMTEHFKRSFTDVKHRWGALKRKTTHSRNPKRSSTTL

SEQ ID NO: 3 - DNA, 3237 bp, Saccharomyces cerevisiae
  source 1..3237: mol_type = genomic DNA
atggacaacaaaagactgtataacggtaacctatccaacattcctgaggttatagatcctggaattacaattccaatttatgaagaggatattagaaatgatacacgcatgaatacaaatgctcgttctgtgagagtctctgacaagagaggtcgttctagctccacttccccacagaaaattggctcctatcgtactcgtgcgggcaggttttcagatactctaaccaatctactcccttcgattagcgctaagctacaccattcaaagaagagtacccctgtagttgttgtgcctcctacgagcagtactccagacagtttaaattccacgacatacgctcccagagtgtctagcgactcttttacggtggctactccgctgtcattacagtctactacgaccagaacaagaacaagaaataacacagtatcttctcagatcactgcaagttcttctttgactactgatgtgggcaaagccacgagtgcaaatatttggagtgctaatgcagaaagtaatacttcttccagtcctttgtttgattaccctctcgccacttcttattttgagccattgactagattcaagtccactgataattatacgctgcctcaaacagctcaactaaacagttttctggagaaaaatgggaatccaaacatatggtcgagtgcgggaaatagtaacactgaccaccttaatactccgatagttaacaggcagcgctctcaatctcaatctaccacaaacagggtttataccgacgcgccctactatcaacagcctgcacagaattaccaggttcaagtgcctcctagagttccaaaaagtacttcaatatcccctgttatccttgatgacgtcgatccagcctcgataaattggattacagcaaaccaaaaagtaccactagtcaatcaaatatcggcgcttttgcccactaacacaatatctatttcaaacgtgtttccattacagcctactcaacaacatcaacaaaacgctgtaaatttgacgagtacttccttagcgactttatgttcacagtatggtaaagtgctttctgcgaggacactaagaggcctgaatatggcattagtagagttttccacagttgaaagtgcaatatgtgctttggaagctctacaaggtaaggaactgtctaaagtaggggcaccaagtacggtatctttcgcgcgagtcttacccatgtatgaacaacctcttaacgttaatggcttcaacaacactcccaagcaaccgctgttgcaagaacagttgaatcacggggtgctcaattatcagctgcaacaatcgctacaacagccggagctacaacaacaaccaacttcatttaatcaaccaaatcttacatattgtaatcccactcagaatttgagccatctgcaattatcttcaaacgaaaatgaaccttaccctttcccgttgcctccaccatcattatcggatagtgaaaaggatatattacacactataagttcttttaagttagaatatgatcatctagagttgaatcatctcttgcagaatgccttgaaaaataaaggagtgtctgatacaaattattttggtcccctgcctgagcacaactccaaagttccaaagagaaaggatacgtttgatgcaccaaaattgcgggaactgagaaagcagtttgactcgaactcattatctactatagaaatggagcaattggcaatcgtcatgctggaccaattaccagaattaagttcagactatttgggtaataccgttattcaaaaattgtttgaaaactcgtcaaatatcatcagggatattatgttgaggaaatgtaataaatatctcacatctatgggtgttcacaaaaacggtacttgggtttgtcagaagatcatcaaaatggcaaacacgccaaggcagataaatcttgtaacttcaggtgtaagcgattactgtactccactcttcaacgaccagtttggaaattatgttattcaaggtattttaaagtttggttttccctggaatagtttcatttttgaaagcgttttatctcatttttggacaatagttcaaaacaggtatggatctcgtgctgtaagggcttgtctagaagcagattccattattacccaatgccaattgctgactatcacttcactgataattgtgctttctccttatctagctacggatacgaatggcactttactgatcacttggctattggatacttgcactttacctaacaagaacttgattttatgtgataaactggtaaataaaaatctggttaaactatgttgtcataaattaggctctcttactgtcttaaagattctcaatttaagaggtggtgaagaagaggcactgtcgaagaataaaattatccatgcaatatttgatggtccaatttctagtgattctatcttgttccagatactagatgagggtaattatggtccaacatttatttataaggttttaacctcgagaattcttgacaatagcgttagagatgaagctataacaaagattcgtcaactgattttaaattccaatatcaatttacaaagtcgccagttactggaggaagttgggctatcatcagcaggaatatcacccaaacaatcctccaagaatcatcgtaaacagcatcctcaggggtttcattctcctggtcgcgccagaggcgtttctgtttccagtgtgagaagttcaaattcaaggcataatagcgttatacaaatgaacaatgcaggtccaacgccagcattgaactttaacccagccccaatgagtgaaataaattcctacttcaacaatcaacaagtggtgtactccggaaatcaaaatcaaaatcagaacggtaattctaacggattagatgaattgaattcacaatttgattcatttagaattgctaatggaacaaatttatccctacctatagttaacttaccaaatgtaagtaataataataacaattataataattctggttattcgagtcaaatgaatcctctaagccgctcagtgagtcataataacaataataataccaataactacaacaataatgataatgataacaataataataacaataacaataataataataataataataataataataataataataataataataatagtaataatagtaataataataataataatgatacaagtttgtaccgctacaggtcatacggatattag

SEQ ID NO: 4 - protein, 1078 aa, Saccharomyces cerevisiae
MDNKRLYNGNLSNIPEVIDPGITIPIYEEDIRNDTRMNTNARSVRVSDKRGRSSSTSPQKIGSYRTRAGRFSDTLTNLLPSISAKLHHSKKSTPVVVVPPTSSTPDSLNSTTYAPRVSSDSFTVATPLSLQSTTTRTRTRNNTVSSQITASSSLTTDVGKATSANIWSANAESNTSSSPLFDYPLATSYFEPLTRFKSTDNYTLPQTAQLNSFLEKNGNPNIWSSAGNSNTDHLNTPIVNRQRSQSQSTTNRVYTDAPYYQQPAQNYQVQVPPRVPKSTSISPVILDDVDPASINWITANQKVPLVNQISALLPTNTISISNVFPLQPTQQHQQNAVNLTSTSLATLCSQYGKVLSARTLRGLNMALVEFSTVESAICALEALQGKELSKVGAPSTVSFARVLPMYEQPLNVNGFNNTPKQPLLQEQLNHGVLNYQLQQSLQQPELQQQPTSFNQPNLTYCNPTQNLSHLQLSSNENEPYPFPLPPPSLSDSEKDILHTISSFKLEYDHLELNHLLQNALKNKGVSDTNYFGPLPEHNSKVPKRKDTFDAPKLRELRKQFDSNSLSTIEMEQLAIVMLDQLPELSSDYLGNTVIQKLFENSSNIIRDIMLRKCNKYLTSMGVHKNGTWVCQKIIKMANTPRQINLVTSGVSDYCTPLFNDQFGNYVIQGILKFGFPWNSFIFESVLSHFWTIVQNRYGSRAVRACLEADSIITQCQLLTITSLIIVLSPYLATDTNGTLLITWLLDTCTLPNKNLILCDKLVNKNLVKLCCHKLGSLTVLKILNLRGGEEEALSKNKIIHAIFDGPISSDSILFQILDEGNYGPTFIYKVLTSRILDNSVRDEAITKIRQLILNSNINLQSRQLLEEVGLSSAGISPKQSSKNHRKQHPQGFHSPGRARGVSVSSVRSSNSRHNSVIQMNNAGPTPALNFNPAPMSEINSYFNNQQVVYSGNQNQNQNGNSNGLDELNSQFDSFRIANGTNLSLPIVNLPNVSNNNNNYNNSGYSSQMNPLSRSVSHNNNNNTNNYNNNDNDNNNNNNNNNNNNNNNNNNNNNNNNSNNSNNNNNNDTSLYRYRSYGY

SEQ ID NO: 5 - DNA, 400 bp, Saccharomyces cerevisiae
  source 1..400: mol_type = genomic DNA
cacataacactgagtgagccgcctcgtgcgttgcgttagaaaaagttaacaaaagtgaaaaaaagcaaggatattttcttgcggaaaaaaaaaaaataaaaattctaaatgtacggattctttgaaaatttgaacagtcttagtttcgtttgagatcttttggtaacccggctgttgtcccaaaaaagaactaggtaaaaaaaaattgtgaaatagtatgaaaccacccctcgaaagaacaagcgatggtgcatatactactgaatggcacagagcaacggcataacttactttcctctatagtaaccattcaattatttgttgctagcttaagtgtatattcattgttagagagatatatctactatcgctgtcccttgctactgttttctgaattctg

SEQ ID NO: 6 - DNA, 24 bp, Saccharomyces cerevisiae
  source 1..24: mol_type = genomic DNA
gatcgttgtttgaagccctttaaa

SEQ ID NO: 7 - DNA, 24 bp, Saccharomyces cerevisiae
  source 1..24: mol_type = genomic DNA
aaactttaaagggcttcaaacaac

SEQ ID NO: 8 - DNA, 24 bp, Saccharomyces cerevisiae
  source 1..24: mol_type = genomic DNA
cgaggatcgtggacacggagctgc

SEQ ID NO: 9 - DNA, 34 bp, Saccharomyces cerevisiae
  source 1..34: mol_type = genomic DNA
gataataccatatattaaactactgtcaattggc

SEQ ID NO: 10 - DNA, 47 bp, Saccharomyces cerevisiae
  source 1..47: mol_type = genomic DNA
agtttaatatatggtattatcgagtcattgcgtttgtatgacatttc

SEQ ID NO: 11 - DNA, 85 bp, Saccharomyces cerevisiae
  source 1..85: mol_type = genomic DNA
tgctattctgaccaatcagcagctattacttcgcgccgggtggccctaacccgtttttttccaaataaaagcaaacaggcacgtg

SEQ ID NO: 12 - DNA, 22 bp, Saccharomyces cerevisiae
  source 1..22: mol_type = genomic DNA
ctgctgattggtcagaatagca

SEQ ID NO: 13 - DNA, 25 bp, Saccharomyces cerevisiae
  source 1..25: mol_type = genomic DNA
gcgcgatactacagttattgtgtag

SEQ ID NO: 14 - DNA, 24 bp, Saccharomyces cerevisiae
  source 1..24: mol_type = genomic DNA
gatctcgcgtatttcgtctcgctc

SEQ ID NO: 15 - DNA, 24 bp, Saccharomyces cerevisiae
  source 1..24: mol_type = genomic DNA
aaacgagcgagacgaaatacgcga

SEQ ID NO: 16 - DNA, 20 bp, Saccharomyces cerevisiae
  source 1..20: mol_type = genomic DNA
gacatggaggcccagaatac

SEQ ID NO: 17 - DNA, 21 bp, Saccharomyces cerevisiae
  source 1..21: mol_type = genomic DNA
cagtatagcgaccagcattca

SEQ ID NO: 18 - DNA, 21 bp, Saccharomyces cerevisiae
  source 1..21: mol_type = genomic DNA
gttggttgttgttgtagctcc

SEQ ID NO: 19 - DNA, 23 bp, Saccharomyces cerevisiae
  source 1..23: mol_type = genomic DNA
gttgttggatttccaacttcttg

SEQ ID NO: 20 - DNA, 24 bp, Saccharomyces cerevisiae
  source 1..24: mol_type = genomic DNA
gatctgttaactatcggagtatta

SEQ ID NO: 21 - DNA, 24 bp, Saccharomyces cerevisiae
  source 1..24: mol_type = genomic DNA
aaactaatactccgatagttaaca

SEQ ID NO: 22 - DNA, 21 bp, Saccharomyces cerevisiae
  source 1..21: mol_type = genomic DNA
ctgcctgttaactatcggagt

SEQ ID NO: 23 - DNA, 50 bp, Saccharomyces cerevisiae
  source 1..50: mol_type = genomic DNA
actccgatagttaacaggcaggcgtctcaatctcaatctaccacaaacag

SEQ ID NO: 24 - DNA, 50 bp, Saccharomyces cerevisiae
  source 1..50: mol_type = genomic DNA
actccgatagttaacaggcagtgctctcaatctcaatctaccacaaacag

SEQ ID NO: 25 - DNA, 50 bp, Saccharomyces cerevisiae
  source 1..50: mol_type = genomic DNA
actccgatagttaacaggcaggactctcaatctcaatctaccacaaacag

SEQ ID NO: 26 - DNA, 50 bp, Saccharomyces cerevisiae
  source 1..50: mol_type = genomic DNA
actccgatagttaacaggcaggagtctcaatctcaatctaccacaaacag

SEQ ID NO: 27 - DNA, 50 bp, Saccharomyces cerevisiae
  source 1..50: mol_type = genomic DNA
actccgatagttaacaggcaggggtctcaatctcaatctaccacaaacag

SEQ ID NO: 28 - DNA, 50 bp, Saccharomyces cerevisiae
  source 1..50: mol_type = genomic DNA
actccgatagttaacaggcagctctctcaatctcaatctaccacaaacag

SEQ ID NO: 29 - DNA, 50 bp, Saccharomyces cerevisiae
  source 1..50: mol_type = genomic DNA
actccgatagttaacaggcagatgtctcaatctcaatctaccacaaacag

SEQ ID NO: 30 - DNA, 50 bp, Saccharomyces cerevisiae
  source 1..50: mol_type = genomic DNA
actccgatagttaacaggcagccctctcaatctcaatctaccacaaacag

SEQ ID NO: 31 - DNA, 50 bp, Saccharomyces cerevisiae
  source 1..50: mol_type = genomic DNA
actccgatagttaacaggcagaactctcaatctcaatctaccacaaacag

SEQ ID NO: 32 - DNA, 50 bp, Saccharomyces cerevisiae
  source 1..50: mol_type = genomic DNA
actccgatagttaacaggcagtcgtctcaatctcaatctaccacaaacag

SEQ ID NO: 33 - DNA, 50 bp, Saccharomyces cerevisiae
  source 1..50: mol_type = genomic DNA
actccgatagttaacaggcagtggtctcaatctcaatctaccacaaacag

(19) United States
(12) Patent Application Publication
(10) Pub. No.: US 2026/0226512 A1
(43) Pub. Date: Aug. 6, 2026

(54) METHOD FOR IMPROVING RATE OF XYLOSE AND ARABINOSE UTILIZATION IN SACCHAROMYCES CEREVISIAE

(71) Applicants: SHANGHAI RESEARCH AND DEVELOP CENTER OF INDUSTRIAL BIOTECHNOLOGY, Shanghai (CN); CAS CENTER FOR EXCELLENCE IN MOLECULAR PLANT SCIENCES, CHINESE ACADEMY OF SCIENCES, Shanghai (CN)

(72) Inventors: Sheng YANG, Shanghai (CN); Fenghul QIAN, Shanghai (CN); Yu JIANG, Shanghai (CN); Yiwen ZHANG, Shanghai (CN); Junjie YANG, Shanghai (CN)

(73) Assignees: SHANGHAI RESEARCH AND DEVELOP CENTER OF INDUSTRIAL BIOTECHNOLOGY, Shanghai (CN); CAS CENTER FOR EXCELLENCE IN MOLECULAR PLANT SCIENCES, CHINESE ACADEMY OF SCIENCES, Shanghai (CN)

(21) Appl. No.: 19/153,170

(22) PCT Filed: Feb. 1, 2024
(86) PCT No.: PCT/CN2024/075298

§ 371 Date: Aug. 1, 2025

(30) Foreign Application Priority Data
Feb. 3, 2023 (WO) PCT/CN2023/074360

(51) Int. Cl. C12P 7/56; C07K 14/395; C12N 9/12; C12N 9/90; C12N 15/52; C12P 5/02; C12P 7/16; C12P 7/46

(52) U.S. Cl. CPC C12P 7/56; C07K 14/395; C12N 9/1205; C12N 9/90; C12N 15/52; C12P 5/026; C12P 7/16; C12P 7/46; C12Y 207/01016; C12Y 503/01004; C12Y 503/01005

(57) ABSTRACT

The provided is a method for improving the rate of glucose, xylose and arabinose utilization in Saccharomyces cerevisiae, comprising the following steps: using Saccharomyces cerevisiae with the ability to convert xylose and/or arabinose into ethanol, lactic acid, butanedioic acid, farnesene or isobutanol as a chassis cell to make the following mode of modifications in its genome: A. a mutation in the TRK1 gene for the potassium ion intracellular transporter protein, resulting in an increase in intracellular potassium ion/sodium ion ratio in Saccharomyces cerevisiae, and/or B. a mutation in the PUF2 gene for mRNA-binding protein or PUF2 gene knockout, resulting in PUF2 inactivation, deletion, reduced function, or down-regulated mRNA translation strength. The recombinant Saccharomyces cerevisiae constructed can utilize the cellulase hydrolysate comprising glucose, xylose and/or arabinose to produce ethanol, lactic acid, butanedioic acid, farnesene or isobutanol by fermentation, and has broad application prospects.

### TECHNICAL FIELD

The present invention belongs to the field of genetic engineering, and relates to methods for improving the rate of xylose and arabinose utilization in Saccharomyces cerevisiae, and a genetically engineered Saccharomyces cerevisiae strain for producing ethanol, lactic acid, butanedioic acid, farnesene or isobutanol by means of metabolizing glucose, xylose and/or arabinose.

### BACKGROUND ART

Fuel ethanol is a widely used renewable fuel component. The first-generation ethanol production process in which grain raw materials such as corn or sugar cane are traditionally used for fermentation by Saccharomyces cerevisiae has the problem of “competing with people for grain and with grain for land”. The second-generation ethanol production process using non-grain lignocellulose such as agricultural waste as raw materials is more sustainable and has less greenhouse gas emissions.

Second-generation bioethanol synthesized from lignocellulose is the most prominent biofuel and is considered to be one of the most important chemicals obtained from biomass. The production of second-generation bioethanol is known to require the following major steps: (1) pretreatment to break the structure of lignocellulose which is difficult to degrade; (2) hydrolysis of cellulose and hemicellulose into fermentable sugars; (3) fermentation by microorganisms (generally Saccharomyces cerevisiae) to produce ethanol; (4) dehydration and distillation of bioethanol. Dilute acid pretreatment is an economical pretreatment method approved by the National Renewable Energy Laboratory of the U.S. Department of Energy, and is a common economic and effective pretreatment method. The pretreatment process produces a variety of by-products that inhibit enzymes and microorganisms, including sulfuric acid, acetic acid, formic acid, hydroxymethylfurfural, and furfural, the most inhibitory of which is the high concentration of sodium salts, including sodium formate, sodium acetate, and sodium sulfate, caused by the introduction of sodium hydroxide during the neutralization process. After undergoing hydrolysis, cellulose and hemicellulose are hydrolyzed to hexoses (e.g., glucose) and pentoses (e.g., xylose, arabinose), and the inhibitors still exist in the hydrolyzate, and thus remains in the fermentation broth.

Saccharomyces cerevisiae is the microorganism of choice for bioethanol production from lignocellulose, and various genetic modifications have been attempted to improve bioethanol synthesis. It is generally believed that a strain of excellent cellulosic ethanol-producing yeast should have the following characteristics: 1) powerful assimilation of xylose or arabinose; 2) co-utilization of pentoses and hexoses; 3) resistance to inhibitors, tolerance to sodium salts.

At present, the introduction and enhancement of xylose metabolism or arabinose metabolism pathways by means of metabolic engineering can confer the ability to utilize xylose or arabinose on Saccharomyces cerevisiae, which cannot naturally utilize the above-mentioned two sugars. The specific methods comprise introduction of xylose isomerase xylA (e.g., see patent document CN 113736675 A, the content of which is incorporated herein by reference) or arabinose metabolism gene expression cassette araBAD (e.g., see patent document CN 110872596 A, the content of which is incorporated herein by reference); multi-copy integration to enhance the above-mentioned gene expression; multi-copy integration to enhance expression of pentose phosphate pathway genes. However, the sugar utilization efficiency of recombinant Saccharomyces cerevisiae strains still needs to be improved, especially in the high sodium salt environment of cellulose hydrolyzate, and more effective targets for improving sugar utilization or sodium salt tolerance still need to be explored. Saccharomyces cerevisiae, which uses cellulose hydrolysate efficiently, will help to convert cellulose hydrolysate into various products, such as ethanol, lactic acid, butanedioic acid, farnesene or isobutanol, which has great application prospects.

### SUMMARY OF THE INVENTION

The present invention improves on the prior art Saccharomyces cerevisiae that already has the ability to utilize xylose or arabinose to produce ethanol, lactic acid, butanedioic acid, farnesene or isobutanol by fermentation by making the strain more tolerant to a high sodium salt concentration environment such as a sodium ion concentration (e.g., greater than 100 mM), and further improves the strain utilization efficiency of glucose, xylose and arabinose contained in the cellulase hydrolysate to make the strain suitable for the conversion and metabolism of these fermentable sugars as carbon sources to ethanol, lactic acid, butanedioic acid, farnesene or isobutanol in the presence or absence of high concentrations of sodium salts. Our research group found and excavated by adaptive evolution and forward genetics analysis more effective targets for sugar utilization, such as TRK1 and PUF2 genes. By means of modifying the genome of these Saccharomyces cerevisiae strains, it was confirmed that some gene mutations improved the strain's xylose and arabinose utilization. This important discovery forms the basis of the present invention and is of great significance for obtaining an engineered strain of Saccharomyces cerevisiae with a higher ability to convert xylose and arabinose to ethanol, lactic acid, butanedioic acid, farnesene or isobutanol at high concentrations of sodium salt. Specifically, the technical solution of the present invention is as follows.

A method for improving xylose and arabinose utilization of Saccharomyces cerevisiae; or a method for improving glucose and arabinose utilization of Saccharomyces cerevisiae; or a method for improving glucose, xylose and arabinose utilization of Saccharomyces cerevisiae; or a method for improving sodium salt tolerance of Saccharomyces cerevisiae; or a method for improving fermentation of ethanol, lactic acid, butanedioic acid, farnesene or isobutanol; said method comprising the following steps:
fermenting said Saccharomyces cerevisiae with the ability to convert xylose and/or arabinose into ethanol, lactic acid, butanedioic acid, farnesene or isobutanol, and optionally recovering the fermentation product;
wherein said Saccharomyces cerevisiae comprises one or both of the following modifications in its genome:
A. a mutation in an endogenous TRK1 gene for the potassium ion intracellular transporter protein, resulting in an increase in intracellular potassium/sodium ratio in Saccharomyces cerevisiae, i.e. an increase in intracellular potassium and/or a decrease in intracellular sodium in Saccharomyces cerevisiae, and/or
B. a mutation in an endogenous PUF2 gene for mRNA-binding protein or PUF2 gene knockout, resulting in PUF2 inactivation, deletion, reduced function, or down-regulated mRNA translation strength.

The nucleotide sequence of the TRK1 gene is SEQ ID NO: 1 in the sequence listing, and the encoded amino acid sequence is SEQ ID NO: 2.

The nucleotide sequence of the PUF2 gene is SEQ ID NO: 3 in the sequence listing, and the encoded amino acid sequence is SEQ ID NO: 4.

For example, the above-mentioned Saccharomyces cerevisiae cell may be an alcohol-producing yeast (e.g., ethanol), such as Angel yeast (AQ), CICC1300, CICC1308, CGMCC2.4705, CGMCC2.4706, and CGMCC2.4804.

The mutation in the endogenous TRK1 gene in the above-mentioned mode A can be selected from mutations (e.g., substitutions) corresponding to positions 764, 905, 988, 1170 and/or 1182 of SEQ ID NO: 2 (e.g., D1182, A1170, L988, P905 and/or E764).

Further, the mutation in the endogenous TRK1 gene in the above-mentioned mode A can be selected from the following mutations: D1182Y, A1170T, A1170M, A1170V, L988S, L988F, P905H, P905S, E764K and a combination of two or more thereof corresponding to SEQ ID NO: 2. In one embodiment, the mutant encodes a variant having at least 65%, e.g., at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the polypeptide of SEQ ID NO: 2.

The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:
(Identical Residues×100)/(Length of Alignment−Total Number of Gaps in Alignment) For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:
(Identical Deoxyribonucleotides×100)/(Length of Alignment−Total Number of Gaps in Alignment).

In one embodiment, the mutated TRK1 gene coding sequence has at least 65%, e.g., at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the TRK1 gene coding sequence of SEQ ID NO: 1. In one embodiment, the endogenous TRK1 gene coding sequence being mutated is the TRK1 gene coding sequence of SEQ ID NO: 1.

The mutation in the endogenous PUF2 gene in the above-mentioned mode B can be selected from one of the following mutations or from a combination of two or more of the following mutations:
B-1. mutation corresponding to position 243 of SEQ ID NO: 4 (e.g., R243);
B-2. PUF2 gene disruption (e.g. knockout/inactivation/downregulation);
B-3. deletion of A base at position 305 upstream of the PUF2 gene.

The nucleotide sequence of upstream 400 bp of the PUF2 gene is SEQ ID NO: 5 in the sequence listing.

Further, the mutation in the endogenous PUF2 gene in the above-mentioned mode B-1 can be selected from, e.g., the following mutations: R243A, R243C, R243D, R243E, R243G, R243L, R243M, R243N, R243P, R243S or R243W corresponding to SEQ ID NO: 4.

In one embodiment, the mutant PUF2 gene encodes a variant having at least 65%, e.g., at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the polypeptide of SEQ ID NO: 4.

In one embodiment, the mutated PUF2 gene coding sequence has at least 65%, e.g., at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the PUF2 gene coding sequence of SEQ ID NO: 3. In one embodiment, the endogenous PUF2 gene coding sequence being mutated is the PUF2 gene coding sequence of SEQ ID NO: 3.

The gene disruption includes gene knockout, deletion or inactivation.

The gene knockout/deletion can be implemented by gene editing techniques known in the art, such as homologous double exchange, TALEN system, CRISPR-Cas9 system, CRISPR-Cpf1 system, CRISPR-Cas12 system, CRISPR-BEST system, and CRISPRi.

The gene inactivation is implemented by modes selected from the group consisting of: complete deletion of nucleotide sequence, partial deletion of nucleotide sequence, gene mutation, and in-frame mutation of stop codons.

The down-regulated expression of gene can be implemented by techniques known in the art, such as protein ubiquitination modification, RNA interference (RNAi), structural changes in expression systems, negative regulation at the transcriptional level, negative regulation at the post-transcriptional level, decreased gene transcription, decreased gene translation, enhanced protein degradation, and a combination of two or more thereof.

Preferably, the chassis cell is a diploid Saccharomyces cerevisiae.

The second aspect of the present invention provides a recombinant Saccharomyces cerevisiae constructed by the method as described.

In one embodiment, the exogenous xylose isomerase gene xylA and/or the arabinose metabolism-related gene expression cassette araBAD are/is also introduced into the recombinant Saccharomyces cerevisiae host cell, and the xylulose kinase gene and the pentose phosphate pathway gene (XKS1+PPP) are enhanced. The xylA gene may be a xylose isomerase gene XylA derived from Piromyces with a nucleotide sequence of SEQ ID NO: 1 disclosed in patent document CN 113736675 A (the content of which is incorporated herein by reference), or xylose isomerase gene RuXylA derived from bovine rumen metagenome with a nucleotide sequence of SEQ ID NO: 2 disclosed in patent document CN 113736675 A; the arabinose metabolism-related gene expression cassette araBAD is arabinose metabolism gene expression cassette AUC disclosed in the patent document CN 110872596 A (the content of which is incorporated herein by reference), including sequentially from upstream to downstream a gene arab expression element with a base sequence of SEQ ID NO: 2 of CN 110872596 A, a gene araA expression element with a base sequence of SEQ ID NO: 1 of CN 110872596 A, a gene araD expression element with a base sequence of SEQ ID NO: 3 of CN 110872596 A, a gene GAL2 expression element with a base sequence of SEQ ID NO: 4 of CN 110872596 A and a gene STP2 expression element with a base sequence of SEQ ID NO: 5 of CN 110872596 A.

Preferably, the copy number of the xylA gene in the genome is 30-50, preferably about 50 copies; the copy number of the araBAD gene expression cassette AUC in the genome is 1-12, preferably about 12 copies.

In some embodiments, the above-mentioned recombinant Saccharomyces cerevisiae host cell also contains the gene NFS11492N mutation in the genome disclosed in the patent document CN 113736675 A, and/or gene ISU1 inactivation, and/or gene CCC1 inactivation, so as to increase the cytoplasmic iron concentration.

In some embodiments, XKS1 and PPP genes are also introduced into the genome of the recombinant Saccharomyces cerevisiae. The genome copy numbers of the XKS1 and PPP genes can be, e.g., 2-3.

In some embodiments, the above-mentioned pentose phosphate pathway genes comprise a transaldolase gene TAL1, a ribulose-5-phosphate isomerase gene RPE1, a transketolase gene TKL1 and/or a ribose-5-phosphate isomerase gene RKI1.

Preferably, the above-mentioned recombinant Saccharomyces cerevisiae can also downregulate the Sln1 branch of the HOG-MAPK pathway, including but not limited to disrupting (e.g., knocking out) SSK1 or SSK2, or mutating SSK1 at a position corresponding to 566, 570 or 628 (e.g., A566D, R570M or D628G), or mutating SSK2 at a position corresponding to 1460 (e.g., T1460A).

Further, the above-mentioned recombinant Saccharomyces cerevisiae can also upregulate the cAMP-Ras-PKA pathway, including but not limited to interrupting or knocking out IRA1.

The above-mentioned recombinant Saccharomyces cerevisiae can also produce high-value biochemical products such as lactic acid, butanedioic acid, farnesene or isobutanol by fermentation, not only ethanol.

When the above-mentioned recombinant Saccharomyces cerevisiae is used to produce lactic acid, the Idh gene from Lactobacillus acidophilus ATCC4356 under the control of PGK1 promoter may also be integrated into the genome.

When the above-mentioned recombinant Saccharomyces cerevisiae is used to produce butanedioic acid, genes SDH1, SDH2, IDH1 and IDP1 may also be disrupted (e.g., knocked out).

When the above-mentioned recombinant Saccharomyces cerevisiae is used to produce farnesene, the BFS gene may be overexpressed, the mevalonate pathway of the Saccharomyces cerevisiae host strain may be enhanced, and the sterol synthesis pathway may be weakened/inhibited, wherein the BFS gene may be a gene with a nucleotide sequence of SEQ ID NO: 1 disclosed in patent document CN 111690690 A or a gene with the same function.

When the above-mentioned recombinant Saccharomyces cerevisiae is used to produce isobutanol, the following three endogenous enzymes of valine synthesis pathway may be overexpressed in cytoplasm: acetolactate synthase (Ilv2), acetohydroxyacid reducisomerase (Ilv5) and dihydroxyacid dehydratase (Ilv3); valine synthesis ILV2 in mitochondria may be disrupted (e.g., knocked out); the following competitive pathway genes may be disrupted (e.g., knocked out): genes BDH1 and BDH2 in 2,3-butanediol pathway, genes LEU4 and LEU9 in leucine pathway, gene ECM31 in pantothenic acid pathway and ILV1 in isoleucine pathway; the following genes may be disrupted (e.g., knocked out): alcohol dehydrogenase gene ADH1, 3-phosphoglyceratede hydrogenase gene GPD1 and GPD2, and aldehyde dehydrogenase gene ALD6.

The third aspect of the present invention provides the use of the above-mentioned recombinant Saccharomyces cerevisiae in ethanol, lactic acid, butanedioic acid, farnesene or isobutanol production by fermentation.

For example, the recombinant Saccharomyces cerevisiae can utilize fermentable sugars in the cellulase hydrolysate as a carbon source including glucose, xylose and/or arabinose for fermentation.

The present invention constructs a recombinant Saccharomyces cerevisiae that can efficiently utilize the fermentable sugars such as glucose, xylose and arabinose in the cellulase hydrolysate by genetic engineering. The strain can also be cultured and proliferated normally under high sodium salt concentration, such as under sodium ion concentration greater than 80 mM, even greater than 100 mM, and can convert glucose, xylose and arabinose in the cellulase hydrolysate into ethanol, lactic acid, butanedioic acid, farnesene or isobutanol, which has broad prospects for industrial application.

### DETAILED DESCRIPTION OF EMBODIMENTS

In the study of the adaptive evolution of xylose- or arabinose-utilizing competent Saccharomyces cerevisiae strains under high sodium salt concentration, our research group used comparative genome analysis and forward genetics analysis to excavate more effective targets beneficial for sugar utilization, including TRK1 gene and PUF2 gene. The forward mutation of these genes can improve the utilization efficiency of xylose and arabinose in Saccharomyces cerevisiae strains in the presence or absence of high sodium salt concentration, and/or enhance the adaptability of Saccharomyces cerevisiae strains to high concentration of sodium salt environment, which is conducive to the rational reconstruction of recombinant Saccharomyces cerevisiae strains that can ferment xylose and arabinose.

In some embodiments, the term “enhance” or “increase” may mean an increase of at least 10% compared to a reference level, such as a chassis cell/starting strain level, e.g, an increase of at least about 20%, or at least about 30%, or at least about 40%, or at least about 50%, or at least about 60%, or at least about 70%, or at least about 80%, or at least about 90%, or up to and including an increase of 100%, or any increase between 10%-100% compared to a reference level, or at least about 2-fold, or at least about 3-fold, or at least about 4-fold, or at least about 5-fold, or at least about 10-fold, or any increase between 2-fold to 10-fold or more compared to a reference level.

The terms “recombinant Saccharomyces cerevisiae”, “Saccharomyces cerevisiae engineered strains”, “genetically engineered strains” and “evolutionary strains” herein have the same meaning, and all refer to the Saccharomyces cerevisiae strains with improved tolerance to high concentration of sodium salts and xylose/arabinose utilization rate, of which chassis strains (starting strains) that have been genetically modified.

TRK1 is a potassium ion intracellular transporter; PUF2 is an mRNA-binding protein that regulates the translation strength of a batch of mRNAs. Neither of TRK1 and PUF2 belongs to metabolic pathway enzymes. However, mutations in TRK1 and/or PUF2 can significantly affect the metabolism of glucose, xylose and/or arabinose in Saccharomyces cerevisiae.

Herein, for the convenience of description, a certain protein such as TRK1 is sometimes mixed with the name of its encoding gene (DNA), and those skilled in the art should understand that they represent different substances in different situations of description. Those skilled in the art can easily understand the meaning according to the background and the context. For the description of mutation, it refers to the abbreviations of amino acids, and those skilled in the art should understand the corresponding amino acids. For example, for TRK1, it refers to the protein when used to describe the function or class of potassium ion transporter; when described as a gene, it refers to the gene encoding the TRK1 protein. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine(S) with phenylalanine (F), respectively. For example, the gene mutation TRK D1182Y refers to the gene mutation resulting in a mutant of TRK1-encoding a variant with the substitution D1182Y.

As used herein, the term “or” sometimes means “and/or”, and the term “or” sometimes means “and/or”. The term “and/or” as used in phrases such as “A and/or B” is intended to include both A and B; A or B; A (alone); and B (alone). Likewise, the term “and/or” as used in phrases such as “A, B and/or C” is intended to encompass each of the following embodiments: A, B and C; A, B or C; A or C; A or B; B or C; A and C; A and B; B and C; A (alone); B (alone); and C (alone).

Gene integration or knockout of recombinant Saccharomyces cerevisiae genome can be implemented by means of gene editing techniques. The homologous double exchange, CRISPR-Cas9 system, CRISPR-Cpf1 system, CRISPR-Cas related transposable system, INTEGRATE system or CAST system can be used in the above-mentioned gene editing techniques. Among them, INTEGRATE system refers to the gene editing tool developed by Sam Sternberg research group (Insertion of transposable elements by guide RNA-assisted targeting); CAST system refers to a gene editing tool (CRISPR-associated transposase) developed by Feng Zhang Research Group.

The present invention will be further described below in conjunction with specific examples. It should be understood that these examples are for illustrative purposes only and are not intended to limit the invention. In addition, it should be understood that after reading the concept of the present invention, various modifications or adjustments made by those skilled in the art should fall within the protection scope of the present invention, and these equivalent forms also belong to the defined scope of the appended claims herein.

The addition amount, content and concentration of various substances as referred herein, wherein the percentage content, unless otherwise specified, refers to the mass percentage content.

### EXAMPLES

### Materials and Methods

The primer synthesis and sequencing herein were completed by Beijing Tsingke Biotechnology Co., Ltd.

The molecular biology experiments herein, including plasmid construction, enzyme digestion, competent cell preparation, transformation, etc., are carried out, mainly referring to “Molecular Cloning: A Laboratory Manual (Third Edition)”, edited by J. Sambrook, D. W. Russell (US), translated by Peitang Huang et al., Science Press, Beijing, 2002. Such as the transformation method of the competent cell and the preparation method of the competent cell both refer to “Molecular Cloning: A Laboratory Manual” (third edition) chapter 1 page 96. The specific experiment conditions can be determined by simple tests if necessary.

### Main Media:

LB medium: 5 g/L yeast extract, 10 g/L tryptone, 10 g/L sodium chloride. (LB solid medium with the addition of 20 g/L agar powder.).

YPD medium contains yeast extract 10 g/L, peptone 20 g/L and glucose 20 g/L.

YPDX medium contains yeast extract 10 g/L, peptone 20 g/L, glucose 80 g/L, and xylose 40 g/L.

YPDXI medium is YPDX supplemented with sodium formate 2.92 g/L, sodium acetate 4.1 g/L and sodium sulfate 14.2 g/L.

YPDXA medium is YPDX supplemented with 20 g/L arabinose.

YPDXAI medium is YPDXI supplemented with 20 g/L arabinose.

Diploid Saccharomyces cerevisiae strains CIBT7850, CIBT7856, CIBT7860, CIBT7862, CIBT7863, etc. were constructed and deposited by our research group, and the CRISPR-Cas9 gene editing tool plasmids were constructed and deposited by our research group. Any unit or individual can obtain these strains and plasmids for verification of the present invention, but shall not be used for other purposes, including scientific research and teaching, without the permission of our research group.

### Example 1: Construction of Xylose- or Arabinose-Utilizing Saccharomyces cerevisiae Chassis Strains

Saccharomyces cerevisiae strains with the ability of utilizing xylose or arabinose were constructed, referring to the inventor's patent documents CN 110872596 A and CN 113736675 A.

1. Construction of a Ps-xylA Series of Xylose-Utilizing Saccharomyces cerevisiae Chassis Strains

Referring to the methods in the literature (Jessop-Fabre, M. M. et al. EasyClone-MarkerFree: A vector toolkit for marker-less integration of genes into Saccharomyces cerevisiae via CRISPR-Cas9. Biotechnol J11, 1110-1117, doi: 10.1002/biot.201600147 (2016). Babaei, M. et al. Expansion of EasyClone-MarkerFree toolkit for Saccharomyces cerevisiae genome with new integration sites. FEMS Yeast Res 21, doi: 10.1093/Femsyr/foab027 (2021)), in the diploid Saccharomyces cerevisiae Angel yeast (AQ), the Ps-xylA gene expression cassette derived from Piromyces was inserted into one of the delta sites of multiple copies, i.e. 1 copy was integrated, using homologous recombination. In order to enhance the xylulokinase and pentose phosphate pathway genes (XKS1+PPP), the XKS1+PPP tandem gene expression cassette was homozygously inserted into the GRE3 gene, i.e. 2 copies were integrated, using CRISPR-Cas technology. Additional 1 copy of the XKS1+PPP tandem gene expression cassette was integrated into one of the delta sites, and a total of 3 copies were integrated. The pentose phosphate pathway genes comprise transaldolase gene TAL1, ribulose-5-phosphate isomerase gene RPE1, transketolase gene TKL1 and ribose-5-phosphate isomerase gene RKI1. Using the double-copy Ru-xylA expression cassette as a template, 4 copies of Ru-xylA gene expression cassette were iteratively integrated into 14 neutral sites of the diploid Saccharomyces cerevisiae by CRISPR-Cas9 technology, and a total of 56 copies were integrated.

The above-mentioned strains were subjected to NFS11492N mutation, ISU1 knockout or CCC1 knockout, to obtain strains CIBT7850, CIBT7851 and CIBT7852, respectively.

The chassis strain was replaced by CICC1300, and the strains CIBT7853, CIBT7854 and CIBT7855 were obtained according to the same construction method.

2. Construction of a Ru-xylA Series of Xylose-Utilizing Saccharomyces cerevisiae Chassis Strains

Using Angel yeast (AQ) as the starting host, except that the Ps-xylA gene expression cassette derived from Piromyces was substituted with the Ru-xylA gene expression cassette derived from the bovine rumen content metagenomic library, the construction was the same as that in step 1 to obtain NFS11492N mutant, ISU1 knockout or CCC1 knockout strains CIBT7856, CIBT7857 and CIBT7858.

Using CICC1300 as the starting host, except that the Ps-xylA gene expression cassette derived from Piromyces was substituted with the Ru-xylA gene expression cassette derived from the bovine rumen content metagenomic library, the construction was the same as that in step 1 to obtain NFS11492N mutant, ISU1 knockout or CCC1 knockout strains CIBT7859, CIBT7860 and CIBT7861.

3. Construction of Arabinose-Utilizing Saccharomyces cerevisiae Chassis Strains

Using CRISPR-Cas9 technology, 6 sites integrated with the xylA gene expression cassette in CIBT7850 and CIBT7853 in step 1 were substituted with 2 copies of the arabinose metabolism-related gene expression cassette araBAD, and a total of 12 copies were integrated, of which araA was derived from Bacillus licheniformis, and araBD was derived from Escherichia coli. The rest were constructed in the same way to obtain NFS1^{1492N} mutant strains CIBT7862 and CIBT7863.

### Example 2: Construction of Recombinant Saccharomyces cerevisiae Strain with TRK1 Mutation

The primers required for the construction of recombinant Saccharomyces cerevisiae with TRK1 gene mutation are listed in Table 1 below.

TABLE 1
PCR primer sequences for TRK1 gene mutation

| Primer | |
|---|---|
| name | sequence (5′→3′) |
| L33n20F | GATCGTTGTTTGAAGCCCTTTAAA |
| | (SEQ ID NO: 6) |
| L33n20R | AAACTTTAAAGGGCTTCAAACAAC |
| | (SEQ ID NO: 7) |
| L33UF | CGAGGATCGTGGACACGGAGCTGC |
| | (SEQ ID NO: 8) |
| L33UR | GATAATACCATATATTAAACTACT |
| | GTCAATTGGC (SEQ ID NO: 9) |
| TRK1ovF | AGTTTAATATATGGTATTATCGAG |
| | TCATTGCGTTTGTATGACATTTC |
| | (SEQ ID NO: 10) |
| TRK1ovR | TGCTATTCTGACCAATCAGCAGCT |
| | ATTACTTCGCGCCGGGTGGCCCTA |
| | ACCCGTTTTTTTCCAAATAAAAGC |
| | AAACAGGCACGTG |
| | (SEQ ID NO: 11) |
| L33DF | CTGCTGATTGGTCAGAATAGCA |
| | (SEQ ID NO: 12) |
| L33DR | GCGCGATACTACAGTTATTGTGTA |
| | G (SEQ ID NO: 13) |

In Table 1, the “F” of the primer name suffix represents the forward direction; “R” represents reverse direction.

1. Construction of Saccharomyces cerevisiae Genomic TRK1 Mutants

TRK1 mutation in the Saccharomyces cerevisiae genome using the CRISPR-Cas9 tool. Using TRK1ovF/R as a primers, and the DNA sequence of the A1170T or A1170M or A1170V mutant, or the D1182Y, P905H, P905S, L988S or E764K mutant of wild-type gene TRK1 or TRK1 as a template, the repair template containing the corresponding TRK1-related genes was obtained by PCR. Using L33UF/R or L33DF/R as a primer pair and Angel yeast genome as a template, the upstream and downstream homologous arms of L33 site were obtained by PCR respectively. Using L33UF and L33DR as primers, and the above-mentioned three fragments as mixed templates, a repair template containing homologous arms and TRK1 sequence was obtained by PCR. The specific PCR method is as follows:

### PCR Reaction System (50 μL):

| | | |
|---|---|---|
| 10 × KOD buffer | 5 | μL |
| dNTP (2.5 mM) | 4 | μL |
| MgCl₂ (25 mM) | 3 | μL |
| KOD Plus | 1 | μL |
| Template | 0.5 | μL |
| Forward primer (10 μM) | 4 | μL |
| Reverse primer (10 μM) | 4 | μL |
| +ddH₂O making up to | 50 | μL |

### PCR Reaction Conditions:

| 94° C. | 5 | min |
|---|---|---|
| 94° C. | 30 | s |
| 55° C. | 30 | s |
| 68° C. | 2 min (totally 30 cycles from the condition at 94° C. for 30 s) | |
| 68° C. | 10 | min |
| 16° C. | 10 | min |

KOD Plus kit was purchased from Toyobo (Shanghai) Biotechnology Co., Ltd.

Construction of the psgRNA-L33 plasmids required for gene editing. First, a DNA fragment containing an intervening sequence was obtained by annealing the primer L33n20F/R, and then ligated with the psgRNA plasmid by T4 ligation reaction. Finally, the competent E. coli cells were transformed, and the correct psgRNA-L33 plasmids were obtained by colony PCR and sequencing verification.

### Annealing System and Conditions:

| | | |
|---|---|---|
| ddH₂O | 25 | μl |
| T4 ligase buffer | 5 | μl |
| Forward primer (10 μM) | 10 | μl |
| Reverse primer (10 μM) | 10 | μl |

Heat preservation at 95° C. for 5 min, and the temperature successively decreased by 5° C.-10° C. every minute, and heat preservation at 16° C. for 10 min.

After diluted 20 times, 1 μl of the annealed product was taken for T4 ligation with the psgRNA plasmid digested with 1 μl of the restriction endonuclease BsaI.

### T4 Ligation System and Conditions:

| | | |
|---|---|---|
| T4 ligase buffer | 1 | μl |
| T4 ligase | 1 | μl |
| Intervening sequence fragments | 5 | μl |
| Digested plasmid | 3 | μl |
| 16° C. | 30 | min. |

Using the CRISPR-Cas9 gene editing system, clones were obtained by transferring pHCas9-Nours, psgRNA-L33 plasmids and repair template fragments. Correctly edited strains with wild-type or mutant TRK1 inserted at L33 were obtained by colony PCR and sequencing.

### Example 3: Fermentation Effect of Recombinant Saccharomyces cerevisiae Strain after Genetic Modification of TRK1

3.1 Fermentation of Recombinant Saccharomyces cerevisiae Strains in the Medium Containing Glucose and Xylose with/without High Sodium Salts.

YPDX medium contains yeast extract 10 g/L, peptone 20 g/L, glucose 80 g/L, and xylose 40 g/L.

YPDXI medium is YPDX supplemented with sodium formate 2.92 g/L, sodium acetate 4.1 g/L and sodium sulfate 14.2 g/L.

The cellulose hydrolysate is an enzymatic hydrolysate of dilute acid and steam explosion pretreated corn straw.

Anaerobic fermentation was carried out in anaerobic test tubes containing 3 mL of YPDX(I) or hydrolysate at 30° C., 240 rpm. First, plate colonies or −80° C. cryopreserved glycerol tube strains were inoculated into 3 mL YPDX medium to grow to logarithmic phase, as primary inoculum solution. Then, 100 μL of the primary inoculum solution were transferred to the same fresh medium and grown to logarithmic phase again, as secondary inoculum solution. The culture was centrifuged at 12000 rpm for 1 min to remove the supernatant, the strains were washed once with sterile water, and was inoculated into YPDX, YPDXI or hydrolysate medium at 0.5 g DCW/L. The conversion ratio of OD₆₀₀ to dry cell weight was 1 OD₆₀₀=0.63 g DCW/L. Samples were taken at specific time points during the fermentation process for determination of sugar and product concentrations. The fermentation results are shown in Table 2 and Table 3, and the data are the average values of three parallel groups.

TABLE 2
Fermentation results of recombinant strains in YPDX medium

| | | YPDX 10 h | YPDX 16.5 h |
|---|---|---|---|
| | Mutant with | glucose | xylose |
| | insertion | Consumption | Consumption |
| Chassis strain | of TRK1 | (g/L) | (g/L) |
| CIBT7850 | Not inserted | 29.1 | 15.1 |
| | A1170T | 46.2 | 28.8 |
| | A1170V | 49.2 | 25.5 |
| | A1170M | 47.5 | 26.2 |
| | D1182Y | 42.5 | 23.8 |
| | P905H | 43.5 | 22.6 |
| | P905S | 49.6 | 22.1 |
| | L988S | 50.6 | 22.6 |
| | E764K | 51.2 | 23.7 |
| CIBT7856 | Not inserted | 29.6 | 15.2 |
| | A1170T | 49.2 | 28.9 |
| | A1170V | 48.5 | 25.4 |
| | A1170M | 46.8 | 25.8 |
| | D1182Y | 46.4 | 25.1 |
| | P905H | 49.6 | 24.9 |
| | P905S | 49.4 | 24.5 |
| | L988S | 46.3 | 24.7 |
| | E764K | 48.2 | 23.5 |

As the comparison results in Table 2, for the two chassis strain CIBT7850 and CIBT7856, after mutating D1182Y, A1170T, A1170M, A1170V, L988S, P905H, P905S, and E764K of the TRK1 gene in their genomes, the ability of the recombinant strains to utilize xylose largely improved. The xylose consumption ability of the recombinant strains obtained by mutating A1170 (including A1170T, A1170M and A1170V) of the TRK1 gene of the chassis CIBT7850 was largely improved; the xylose consumption ability of the recombinant strains obtained by mutating A1170T, A1170M and A1170V of the TRK1 gene of the chassis CIBT7856 also significantly improved.

TABLE 3
Fermentation results of recombinant strains in YPDXI medium

| | | YPDXI 14 h | YPDXI 24 h |
|---|---|---|---|
| | Mutant with | glucose | xylose |
| | insertion | Consumption | Consumption |
| Chassis strain | of TRK1 | (g/L) | (g/L) |
| CIBT7850 | Not inserted | 53.9 | 9.5 |
| | A1170T | 69.6 | 14.9 |
| | A1170V | 68.2 | 15.9 |
| | A1170M | 65.9 | 15.6 |
| | D1182Y | 60.1 | 14.6 |
| | P905H | 60.6 | 13.8 |
| | P905S | 64.6 | 14.9 |
| | L988S | 61.2 | 13.7 |
| | E764K | 58.7 | 15.4 |
| CIBT7856 | Not inserted | 53.6 | 9.1 |
| | A1170T | 60.5 | 15.8 |
| | A1170V | 62.5 | 15.2 |
| | A1170M | 61.6 | 15.4 |
| | D1182Y | 78.9 | 16.2 |
| | P905H | 78.1 | 12.9 |
| | P905S | 71.3 | 10.5 |
| | L988S | 60.4 | 14.3 |
| | E764K | 79.1 | 14.9 |
| | L988F | 79.1 | 14.9 |

As the comparison results in Table 3, for the two chassis strains CIBT7850 and CIBT7855, after mutating D1182Y, A1170T, A1170M, A1170V, L988S, P905H, P905S, and E764K of the TRK1 gene in their genomes, the recombinant strains' ability to utilize xylose under high sodium salt concentration has largely improved. 3.2 Fermentation of recombinant Saccharomyces cerevisiae strains in the medium containing glucose, xylose and arabinose with/without high sodium salts.

20 g/L of arabinose were added to the YPDX and YPDXI media in step 3.1 to obtain media YPDXA and YPDXAI, respectively. The other fermentation conditions were the same as that in step 3.1. The fermentation results are shown in Table 4 and Table 10 5, and the data are the average values of three parallel groups.

TABLE 4
Fermentation results of recombinant strains in YPDXA medium

| Chassis | Mutant with insertion | YPDXA 36 h arabinose |
|---|---|---|
| strain | of TRK1 | consumption (g/L) |
| CIBT7862 | Not inserted | 15.2 |
| | A1170T | 18.2 |
| | A1170V | 17.9 |
| | A1170M | 18.0 |
| | D1182Y | 17.2 |
| | P905H | 17.6 |
| | P905S | 16.5 |
| | L988S | 17.6 |
| | E764K | 16.8 |

As the comparison results in Table 4, for the chassis strain CIBT7862, after mutating D1182Y, A1170T, A1170M, A1170V, L988S, P905H, P905S, and E764K of the TRK1 gene in its genome, the ability of the recombinant strain to utilize arabinose in the medium containing xylose and arabinose improved to a certain extent.

TABLE 5
Fermentation results of recombinant strains in YPDXAI medium

| Chassis | Mutant with insertion | YPDXAI 36 h arabinose |
|---|---|---|
| strain | of TRK1 | consumption (g/L) |
| CIBT7862 | Not inserted | 9.6 |
| | A1170T | 14.1 |
| | A1170V | 13.5 |
| | A1170M | 14.0 |
| | D1182Y | 13.1 |
| | P905H | 13.6 |
| | P905S | 13.0 |
| | L988S | 12.9 |
| | E764K | 12.4 |

As the comparison results in Table 4, for the chassis strain CIBT7862, after mutating D1182Y, A1170T, A1170M, A1170V, L988S, P905H, P905S, and E764K of the TRK1 gene in its genome, the ability of the recombinant strain to utilize arabinose under high sodium salt concentration conditions containing xylose and arabinose improved to a certain extent.

### Example 4: Investigation of Intracellular Sodium and Potassium Ion Concentrations of Recombinant Saccharomyces cerevisiae Under Different Salt Concentrations

Saccharomyces cerevisiae anaerobic fermentation was carried out in anaerobic test tubes containing 3 mL of YPDX(I) or cellulose hydrolysate at 30° C., 240 rpm. First, plate colonies or −80° C. cryopreserved glycerol tube strains were inoculated into 3 mL YPDX medium to grow to logarithmic phase, as primary inoculum solution. Then 100 μL of the primary inoculum solution were taken and transferred to the same fresh medium to grow for-h again. Equal amounts of cells were collected, and the intracellular sodium and potassium ion concentrations were determined by atomic absorption spectrometry, as shown in Table 6 and Table 7.

TABLE 6
Intracellular sodium and potassium ion concentration
of recombinant yeast when cultured in YPDX for 16.5 h

| Chassis | Mutant with | Na⁺ concentration | K⁺ concentration |
|---|---|---|---|
| strain | integration of TRK1 | (mg/gDCW) | (mg/g DCW) |
| AQ | Not integrated | 0.765 | 10.15 |
| | A1170T | 0.19 | 12.82 |
| | A1170V | 0.16 | 12.49 |
| | A1170M | 0.19 | 11.26 |
| | D1182Y | 0.21 | 12.23 |
| | P905H | 0.20 | 12.62 |
| | P905S | 0.26 | 11.02 |
| | L988S | 0.23 | 12.23 |
| | E764K | 0.32 | 11.56 |
| CIBT7856 | Not integrated | 0.85 | 10.85 |
| | A1170T | 0.18 | 12.91 |
| | A1170V | 0.17 | 13.02 |
| | A1170M | 0.21 | 12.43 |
| | D1182Y | 0.24 | 12.56 |
| | P905H | 0.23 | 11.93 |
| | P905S | 0.19 | 12.36 |
| | L988S | 0.26 | 12.24 |
| | E764K | 0.24 | 11.97 |

TABLE 7
Intracellular sodium and potassium ion concentration
of recombinant yeast when cultured in YPDXI for 24 h

| Chassis | Mutant with | Na⁺ concentration | K⁺ concentration |
|---|---|---|---|
| strain | integration of TRK1 | (mg/gDCW) | (mg/g DCW) |
| AQ | Not integrated | 5.72 | 8.06 |
| | A1170T | 2.56 | 12.56 |
| | A1170V | 2.94 | 12.41 |
| | A1170M | 3.41 | 12.81 |
| | D1182Y | 3.65 | 13.01 |
| | P905H | 3.24 | 12.68 |
| | P905S | 2.97 | 12.61 |
| | L988S | 3.81 | 12.84 |
| | E764K | 3.56 | 11.98 |
| CIBT7856 | Not integrated | 5.46 | 7.95 |
| | A1170T | 2.84 | 12.42 |
| | A1170V | 2.28 | 13.34 |
| | A1170M | 3.30 | 12.63 |
| | D1182Y | 3.83 | 12.13 |
| | P905H | 2.98 | 11.6 |
| | P905S | 3.45 | 12.51 |
| | L988S | 3.26 | 12.13 |
| | E764K | 3.56 | 11.81 |

As the comparison results of Table 6 and Table 7, for the chassis Angel yeast (AQ) and CIBT7856, after mutating D1182Y, A1170T, A1170M, A1170V, L988S, P905H, P905S, and E764K of the TRK1 gene in its genome, the intracellular potassium ion concentration of the recombinant strains increased to varying degrees, while the sodium ion concentration decreased to varying degrees, and the potassium ion/sodium ion ratio increased more, which proved that the above-mentioned TRK1 gene mutation increased the intracellular potassium ion/sodium ion intake ratio of S. cerevisiae.

### Example 5: Construction of Recombinant Saccharomyces cerevisiae Strain with PUF2 Mutation

The primers required for the construction of recombinant Saccharomyces cerevisiae with PUF2 gene mutation are listed in Table 8 below.

TABLE 8
PCR primer sequences for PUF2 gene mutation

| Primer | sequence (5′→3′) |
|---|---|
| G418n20F | GATCTCGCGTATTTCGTCTCGCTC |
| | (SEQ ID NO: 14) |
| G418n20R | AAACGAGCGAGACGAAATACGCGA |
| | (SEQ ID NO: 15) |
| G418F | GACATGGAGGCCCAGAATAC |
| | (SEQ ID NO: 16) |
| G418R | CAGTATAGCGACCAGCATTCA |
| | (SEQ ID NO: 17) |
| PUF2-600F | GTTGGTTGTTGTTGTAGCTCC |
| | (SEQ ID NO: 18) |
| PUF2-600R | GTTGTTGGATTTCCAACTTCTTG |
| | (SEQ ID NO: 19) |
| pufn20F | GATCTGTTAACTATCGGAGTATTA |
| | (SEQ ID NO: 20) |
| pufn20R | AAACTAATACTCCGATAGTTAACA |
| | (SEQ ID NO: 21) |
| pufovF | CTGCCTGTTAACTATCGGAGT |
| | (SEQ ID NO: 22) |
| pufovAR | ACTCCGATAGTTAACAGGCAGGCGTCTCAATCT |
| | CAATCTACCACAAACAG (SEQ ID NO: 23) |
| pufovCR | ACTCCGATAGTTAACAGGCAGTGCTCTCAATCT |
| | CAATCTACCACAAACAG (SEQ ID NO: 24) |
| pufovDR | ACTCCGATAGTTAACAGGCAGGACTCTCAATCT |
| | CAATCTACCACAAACAG (SEQ ID NO: 25) |
| pufovER | ACTCCGATAGTTAACAGGCAGGAGTCTCAATCT |
| | CAATCTACCACAAACAG (SEQ ID NO: 26) |
| pufovGR | ACTCCGATAGTTAACAGGCAGGGGTCTCAATCT |
| | CAATCTACCACAAACAG (SEQ ID NO: 27) |
| pufovLR | ACTCCGATAGTTAACAGGCAGCTCTCTCAATCT |
| | CAATCTACCACAAACAG (SEQ ID NO: 28) |
| pufovMR | ACTCCGATAGTTAACAGGCAGATGTCTCAATCT |
| | CAATCTACCACAAACAG (SEQ ID NO: 29) |
| pufovPR | ACTCCGATAGTTAACAGGCAGCCCTCTCAATCT |
| | CAATCTACCACAAACAG (SEQ ID NO: 30) |
| pufovNR | ACTCCGATAGTTAACAGGCAGAACTCTCAATCT |
| | CAATCTACCACAAACAG (SEQ ID NO: 31) |
| pufovSR | ACTCCGATAGTTAACAGGCAGTCGTCTCAATCT |
| | CAATCTACCACAAACAG (SEQ ID NO: 32) |
| pufovWR | ACTCCGATAGTTAACAGGCAGTGGTCTCAATCT |
| | CAATCTACCACAAACAG (SEQ ID NO: 33) |

In Table 8, the “F” of the primer name suffix represents the forward direction; “R” represents reverse direction.

1. Replacement of Saccharomyces cerevisiae Genomic PUF2 with G418 Resistance Gene Expression Cassette

First, the DNA sequence including the PUF2 R243 site and the upstream 305 bases in the Saccharomyces cerevisiae genome was replaced with the G418 resistance gene expression cassette using the CRISPR-Cas9 tool, in order to facilitate subsequent gene editing. The PCR of the upstream and downstream homologous arm fragments and the G418 resistance gene expression cassette used the following methods:

### PCR Reaction System (50 μL):

| | | |
|---|---|---|
| 10 × KOD buffer | 5 | μL |
| dNTP (2.5 mM) | 4 | μL |
| MgCl₂ (25 mM) | 3 | μL |
| KOD Plus | 1 | μL |
| Template | 0.5 | μL |
| Forward primer (10 μM) | 4 | μL |
| Reverse primer (10 μM) | 4 | μL |
| +ddH₂O making up to | 50 | μL |

### PCR Reaction Conditions:

| 94° C. | 5 | min |
|---|---|---|
| 94° C. | 30 | s |
| 55° C. | 30 | s |
| 68° C. | 2 min (totally 30 cycles from the condition at 94° C. for 30 s) | |
| 68° C. | 10 | min |
| 16° C. | 10 | min |

KOD Plus kit was purchased from Toyobo (Shanghai) Biotechnology Co., Ltd.

The upstream and downstream homologous arm fragments and G418 resistance gene expression cassette fragment were used as templates, and PUF2-600F/R was used as primers, and the repair template fragments were obtained by the above-mentioned PCR reaction.

Construction of the psgRNA-PUF2 plasmids required for gene editing. First, a DNA fragment containing an intervening sequence was obtained by annealing the primer pufn20F/R, and then ligated with the psgRNA plasmid by T4 ligation reaction. Finally, the competent E. coli cells were transformed, and the correct psgRNA-PUF2 plasmids were obtained by colony PCR and sequencing verification.

### Annealing System and Conditions:

| | | |
|---|---|---|
| ddH₂O | 25 | μl |
| T4 ligase buffer | 5 | μl |
| Forward primer (10 μM) | 10 | μl |
| Reverse primer (10 μM) | 10 | μl |

Heat preservation at 95° C. for 5 min, and the temperature successively decreased by 5° C.-10° C. every minute, and heat preservation at 16° C. for 10 min.

After diluted 20 times, 1 μl of the annealed product was taken for T4 ligation with the psgRNA plasmid digested with 1 μl of the restriction endonuclease BsaI.

### T4 Ligation System and Conditions:

| | | |
|---|---|---|
| T4 ligase buffer | 1 | μl |
| T4 ligase | 1 | μl |
| Intervening sequence fragments | 5 | μl |
| Digested plasmid | 3 | μl |
| 16° C. | 30 | min. |

Using the CRISPR-Cas9 gene editing system, clones were obtained by transferring pH-Cas9-Nours, psgRNA-PUF2 plasmids and repair template fragments. Correctly edited strains were obtained by colony PCR and sequencing.

2. Substitution of G418 resistance gene with PUF2 mutated sequence Take the substitution of C at the R243 site of PUF2 as an example.

Using PUF2-600F and pufovCR, as well as PUF-600R and pufovF as primers, and the genome of Angel yeast strain as the template, the upstream and downstream fragments were amplified by PCR. Then, using PUF2-600F/R as the primer and the upstream and downstream fragments as templates, the repair template fragments were obtained by PCR.

The PCR reaction conditions, the required reagents, and the construction method of the psgRNA-G418 plasmid are the same as that in step 1 of this example.

A correctly edited recombinant Saccharomyces cerevisiae strain can be obtained by CRISPR-Cas9, and the gene editing method is the same as that in step 1 of this example.

### Example 6: Fermentation Effect of Recombinant Saccharomyces cerevisiae Strain after Genetic Modification of PUF2

1. Fermentation of Recombinant Saccharomyces cerevisiae Strains in the Medium Containing Glucose and Xylose.

Anaerobic fermentation was carried out in anaerobic test tubes containing 3 mL of YPDX(I) or cellulose hydrolysate at 30° C., 240 rpm. First, plate colonies or −80° C. cryopreserved glycerol tube strains were inoculated into 3 mL YPDX medium to grow to logarithmic phase, as primary inoculum solution. Then 100 μL of the primary inoculum solution were transferred to the same fresh medium and grown to logarithmic phase again, as fermented inoculum solution. The culture was centrifuged at 12000 rpm for 1 min to remove the supernatant, the strains were washed once with sterile water, and was inoculated into YPDX, YPDXI or cellulose hydrolysate medium at 0.5 g DCW/L. The conversion ratio of OD₆₀₀ to dry cell weight was 1 OD₆₀₀=0.63 g DCW/L. Samples were taken at specific time points during the fermentation process for determination of sugar and product concentrations. The fermentation results are shown in Tables 9-12, and the numbers are the averages based on three parallels. The strains used in Table 9 and Table 10 are PUF2 mutants based on the chassis strain CIBT7856; The strains used in Table 11 and Table 12 are PUF2 mutants based on the chassis strain CIBT7863.

TABLE 9
Fermentation results of recombinant strains in YPDX medium

| | 10 h glucose | 16.5 h |
|---|---|---|
| | consumption | xyloseconsumption |
| PUF2 gene mutation | (g/L) | (g/L) |
| Not inserted (CIBT7856) | 29.6 | 15.2 |
| R243A | 62.1 | 35.9 |
| R243C | 63.4 | 34.9 |
| R243D | 61.5 | 34.2 |
| R243E | 62.3 | 33.1 |
| R243G | 62.6 | 34.2 |
| R243L | 60.2 | 33.0 |
| R243M | 62.6 | 30.5 |
| R243N | 63.1 | 35.1 |
| R243P | 62.1 | 33.2 |
| R243S | 60.5 | 34.5 |
| R243W | 61.9 | 35.2 |
| puf2Δ (deletion) | 62.5 | 33.2 |
| deletion of A at position 305 | 62.9 | 33.2 |
| upstream of the PUF2 | | |

As the comparison results in Table 9, for the chassis strain CIBT7856, after mutating R243A, R243C, R243D, R243E, R243G, R243L, R243M, R243N, R243P, R243S or R243W of the PUF2 gene, or knocking out PUF2, or deletion of A base at position 305 upstream of the PUF2 in its genome, the ability of the recombinant strains to utilize glucose and xylose in the medium containing xylose was doubled.

TABLE 10
Fermentation results of recombinant strains in YPDXI medium

| | 14 h glucose | 24 h |
|---|---|---|
| | consumption | xyloseconsumption |
| PUF2 gene mutation | (g/L) | (g/L) |
| Not inserted (CIBT7856) | 53.6 | 9.1 |
| R243A | 68.4 | 22.3 |
| R243C | 68.1 | 20.4 |
| R243D | 65.1 | 21.2 |
| R243E | 64.0 | 19.1 |
| R243G | 64.7 | 21.2 |
| R243L | 68.3 | 19.0 |
| R243M | 68.5 | 17.1 |
| R243N | 67.1 | 23.1 |
| R243P | 67.5 | 23.3 |
| R243S | 57.6 | 21.5 |
| R243W | 64.5 | 21.8 |
| puf2Δ | 69.0 | 15.8 |
| deletion of A at position 305 | 68.5 | 15.2 |
| upstream of the PUF2 | | |

As the comparison results in Table 10, for the chassis strain CIBT7856, after mutating R243A, R243C, R243D, R243E, R243G, R243L, R243M, R243N, R243P, R243S or R243W of the PUF2 gene, or knocking out PUF2, or deletion of A base at position 305 upstream of the PUF2 in its genome, the ability of the recombinant strain to utilize glucose and xylose under high sodium salt concentration improved to a certain extent.

2. Fermentation of Recombinant Saccharomyces cerevisiae Strains in the Medium Containing Glucose and Arabinose with or without High Sodium Salts.

20 g/L of arabinose were added to the YPDX and YPDXI media in step 1 to obtain media YPDXA and YPDXAI, respectively. The other fermentation conditions were the same as that in step 1. The fermentation results are shown in Table 11 and Table 12, and the data are the average values of three parallel groups.

TABLE 11
Fermentation results of recombinant strains in YPDXA medium

| | 36 h arabinose |
|---|---|
| | consumption |
| PUF2 gene mutation | (g/L) |
| Not inserted (CIBT7863) | 15.2 |
| R243A | 18.2 |
| R243C | 17.9 |
| R243D | 18.0 |
| R243E | 17.2 |
| R243G | 17.6 |
| R243L | 16.5 |
| R243M | 17.6 |
| R243N | 16.8 |
| R243P | 16.2 |
| R243S | 16.8 |
| R243W | 16.4 |
| puf2Δ (deletion) | 16.3 |
| deletion of A at position 305 upstream of the PUF2 | 16.1 |

As the comparison results in Table 11, for the chassis strain CIBT7863, after mutating R243A, R243C, R243D, R243E, R243G, R243L, R243M, R243N, R243P, R243S or R243W of the PUF2 gene, or knocking out PUF2, or deletion of A base at position 305 upstream of the PUF2 in its genome, the ability of the recombinant strains to utilize arabinose improved to a certain extent.

TABLE 12
Fermentation results of recombinant strains in YPDXAI medium

| | 36 h arabinose |
|---|---|
| | consumption |
| PUF2 gene mutation | (g/L) |
| Not mutated (CIBT7863) | 9.6 |
| R243A | 14.1 |
| R243C | 13.5 |
| R243D | 14.0 |
| R243E | 13.1 |
| R243G | 13.6 |
| R243L | 13.0 |
| R243M | 12.9 |
| R243N | 12.4 |
| R243P | 13.5 |
| R243S | 12.6 |
| R243W | 13.2 |
| puf2Δ | 11.8 |
| deletion of A at position 305 upstream of the PUF2 | 11.5 |

As the comparison results in Table 12, for the chassis strain CIBT7863, after mutating R243A, R243C, R243D, R243E, R243G, R243L, R243M, R243N, R243P, R243S or R243W of the PUF2 gene, or knocking out PUF2, or deletion of A base at position 305 upstream of the PUF2 in its genome, the ability of the recombinant strains to utilize arabinose under high sodium salt concentration improved to a certain extent.

### Example 7: TRK1 or PUF2 Mutations Enhance Lactic Acid Production in Saccharomyces cerevisiae

Referring to the gene editing method described in Example 2, in the Saccharomyces cerevisiae strain CIBT7856, the ldh gene from Lactobacillus acidophilus ATCC4356 expressed under the control of a PGK1 promoter and an expression cassette was integrated into a chromosome to obtain a lactic acid producing strain CIBT7865. In CIBT7865, according to the gene editing method described in Example 2, different mutant TRK1 is introduced on chromosome or PUF2 is mutated or knocked out. The colony of the obtained recombinant strain was cultured in a tube containing 2.5 mL YPDX at 220 rpm at 30° C. for 24 h. Then, 2.5 mL cellulose hydrolysate was fed into a shaker at the rate of 4% v/v (200 μL) at 30° C. and 80 rpm for anaerobic fermentation for 72 h. The concentrations of glucose, xylose and lactic acid were detected by means of HPLC. See Table 13 for lactic acid yield of engineering Saccharomyces cerevisiae.

TABLE 13
Lactic acid yield of engineering Saccharomyces cerevisiae

| Chassis | | Yield of lactic acid |
|---|---|---|
| strain | | (g/L) |
| | Mutant with insertion of TRK1 | |
| CIBT7865 | Not integrated | 40 ± 2 |
| | A1170T | 50 ± 3 |
| | A1170V | 48 ± 5 |
| | A1170M | 45 ± 1 |
| | D1182Y | 42 ± 2 |
| | P905H | 49 ± 1 |
| | P905S | 48 ± 3 |
| | L988S | 44 ± 2 |
| | E764K | 45 ± 2 |
| | PUF2 gene mutant | |
| CIBT7865 | Not mutated | 40 ± 2 |
| | R243A | 48 ± 2 |
| | R243C | 49 ± 5 |
| | R243D | 47 ± 1 |
| | R243E | 46 ± 2 |
| | R243G | 45 ± 1 |
| | R243L | 46 ± 3 |
| | R243M | 44 ± 2 |
| | R243N | 47 ± 2 |
| | R243P | 45 ± 2 |
| | R243S | 44 ± 3 |
| | R243W | 45 ± 1 |
| | puf2Δ (deletion) | 45 ± 2 |
| | deletion of A at position 305 | 43 ± 3 |
| | upstream of the PUF2 | |

As the comparison results in Table 13, when mutating A1170T, A1170V, A1170M, D1182Y, P905H, P905S, L988S, and E764K of TRK1 of the genome of the lactic acid-producing chassis strain CIBT7865 in its genome, the lactic acid production ability of the recombinant strains increased to varying degrees. When the genome of lactic acid-producing chassis strain CIBT7865 undergoes deletion mutations of R243A, R243C, R243D, R243E, R243G, R243L, R243M, R243N, R243P, R243S, R243W, sequence deletion, and 305 A upstream of PUF2 of PUF2 gene, the lactic acid production ability of the recombinant strain also increased to varying degrees, indicating that the above-mentioned mutations of TRK1 or PUF2 enhance the ability of Saccharomyces cerevisiae to produce lactic acid.

### Example 8: TRK1 or PUF2 Mutations Enhance Butanedioic Acid Production in Saccharomyces cerevisiae

Referring to the gene editing method described in Example 2, in the Saccharomyces cerevisiae strain CIBT7856, genes SDH1, SDH2, IDH1 and IDP1 were knocked out to obtain the butanedioic acid-producing recombinant Saccharomyces cerevisiae strain CIBT7866. Using CIBT7866 as a chassis strain, according to the gene editing method described in Example 2, different mutant TRK1 is introduced on chromosome or PUF2 is mutated or knocked out. The colony of the obtained recombinant strain was cultured in a tube containing 2.5 mL YPDX at 220 rpm at 30° C. for 24 h. Then, 2.5 mL cellulose hydrolysate was fed into a shaker at the rate of 4% v/v (200 μL) at 30° C. and 220 rpm for anaerobic fermentation for 72 h. The concentrations of glucose, xylose and butanedioic acid were detected by means of HPLC. See Table 14 for butanedioic acid yield of engineering Saccharomyces cerevisiae.

TABLE 14
Butanedioic acid yield of engineering Saccharomyces cerevisiae

| Chassis | | Yield of butanedioic acid |
|---|---|---|
| strain | | (g/L) |
| | Mutant with insertion of TRK1 | |
| CIBT7866 | Not integrated | 3.6 ± 0.2 |
| | A1170T | 4.5 ± 0.3 |
| | A1170V | 4.8 ± 0.5 |
| | A1170M | 4.5 ± 0.1 |
| | D1182Y | 4.2 ± 2 |
| | P905H | 4.0 ± 0.1 |
| | P905S | 4.3 ± 0.3 |
| | L988S | 4.4 ± 0.2 |
| | E764K | 4.2 ± 0.2 |
| | PUF2 gene mutant | |
| CIBT7866 | Not mutated | 3.6 ± 0.2 |
| | R243A | 4.8 ± 0.2 |
| | R243C | 4.9 ± 0.5 |
| | R243D | 4.7 ± 0.1 |
| | R243E | 4.6 ± 0.2 |
| | R243G | 4.5 ± 0.1 |
| | R243L | 4.6 ± 0.3 |
| | R243M | 4.4 ± 0.2 |
| | R243N | 4.5 ± 0.3 |
| | R243P | 4.4 ± 0.3 |
| | R243S | 4.3 ± 0.3 |
| | R243W | 4.5 ± 0.1 |
| | puf2Δ (deletion) | 4.4 ± 0.2 |
| | deletion of A at position 305 | 4.3 ± 0.3 |
| | upstream of the PUF2 | |

As the comparison results in Table 14, when mutating A1170T, A1170V, A1170M, D1182Y, P905H, P905S, L988S, and E764K of TRK1 gene of the genome of the butanedioic acid-producing chassis strain CIBT7866 in its genome, the butanedioic acid production ability of the recombinant strains increased to varying degrees. When the genome of butanedioic acid-producing chassis strain CIBT7866 undergoes deletion mutations of R243A, R243C, R243D, R243E, R243G, R243L, R243M, R243N, R243P, R243S, R243W, sequence deletion, and 305 A upstream of PUF2 of PUF2 gene, the butanedioic acid production ability of the recombinant strains also increased to varying 10 degrees, indicating that the above-mentioned mutations of TRK1 or PUF2 enhance the ability of Saccharomyces cerevisiae to produce butanedioic acid.

### Example 9: TRK1 or PUF2 Mutations Enhance Farnesene Production in Saccharomyces cerevisiae

Referring to the gene editing method described in Example 2, in the β-farnesene genetically engineered strain CIBTS3737 (deposited by Shanghai Industrial Biotechnology Research and Development Center) reported in patent document CN 111690690 A, a TRK1 gene mutation is introduced into a chromosome, or PUF2 is mutated or PUF2 is knocked out.

The colony of the obtained recombinant strain was cultured in a 24-well plate containing 2.5 mL YPDX at 220 rpm at 30° C. for 48 h. Then, 2.5 mL cellulose hydrolysate was fed into a 24-well plate at the rate of 4% v/v (200 μL) (adding 2 wt % galactose and 25 μM CuSO₄) at 220 rpm at 30° C. for 72 h. After fermentation, 0.5 mL of decane was added to the well plate and cultured in a shaker at 220 rpm at 30° C. for 8-12 h. Taking out and standing for 30 min, then centrifugation at 12000 rpm for 10 min, pipetting the decane phase in the upper layer with a pipette, and send same to GC and GC-MS for analysis. The detection method is consistent with the method reported in Example 4 of patent document CN 111690690 A. See Table 15 for β-farnesene yield of engineering Saccharomyces cerevisiae.

TABLE 15
β-farnesene yield of engineering Saccharomyces cerevisiae

| | | Yield of |
|---|---|---|
| | | farnesene |
| Chassis strain | | (mg/L) |
| | Mutant with insertion of TRK1 | |
| CIBTS3737 | Not integrated | 405 ± 31 |
| | A1170T | 455 ± 28 |
| | A1170V | 448 ± 35 |
| | A1170M | 445 ± 18 |
| | D1182Y | 442 ± 28 |
| | P905H | 439 ± 19 |
| | P905S | 438 ± 30 |
| | L988S | 441 ± 23 |
| | E764K | 438 ± 29 |
| | PUF2 gene mutant | |
| CIBTS3737 | Not integrated | 400 ± 31 |
| | A1170T | 440 ± 25 |
| | A1170V | 442 ± 20 |
| | A1170M | 438 ± 27 |
| | D1182Y | 435 ± 35 |
| | P905H | 427 ± 26 |
| | P905S | 434 ± 19 |
| | L988S | 432 ± 26 |
| | E764K | 437 ± 32 |

As the comparison results in Table 15, when mutating A1170T, A1170V, A1170M, D1182Y, P905H, P905S, L988S, and E764K of TRK1 gene of the genome of the farnesene-producing chassis strain CIBT3737 in its genome, the farnesene production ability of the recombinant strains increased to varying degrees. When the genome of farnesene-producing chassis strain CIBT3737 undergoes deletion mutations of R243A, R243C, R243D, R243E, R243G, R243L, R243M, R243N, R243P, R243S, R243W, sequence deletion, and 305 A upstream of PUF2 of PUF2 gene, the farnesene production ability of the recombinant strains also increased to varying degrees, indicating that the above-mentioned mutations of TRK1 or PUF2 enhance the ability of Saccharomyces cerevisiae to produce farnesene.

### Example 10: TRK1 or PUF2 Mutations Enhance Isobutanol Production in Saccharomyces cerevisiae

In Saccharomyces cerevisiae CIBT7856, three endogenous enzymes of valine synthesis pathway are overexpressed in cytoplasm: acetolactate synthase (Ilv2), acetohydroxyacid reducisomerase (Ilv5) and dihydroxyacid dehydratase (Ilv3); valine synthesis ILV2 in mitochondria is destroyed; the following competitive pathway genes are knocked out: genes (BDH1 and BDH2) in 2,3-butanediol pathway, genes (LEU4 and LEU9) in leucine pathway, gene (ECM31) in pantothenic acid pathway and isoleucine (ILV1); alcohol dehydrogenase ADH1 gene, 3-phosphoglyceratede hydrogenase gene GPD1 and GPD2, and aldehyde dehydrogenase gene ALD6 are knocked out to obtain strain CIBT7866. Referring to the gene editing method described in Example 2, a series of recombinant strains were obtained by means of integrating different mutant types of TRK1 or mutation PUF2 in CIBT7866.

The pre-culture and fermentation culture were grown aerobically in a synthetic complete medium (1.7 g/L amino acid-free yeast nitrogen base, 5 g/L ammonium sulfate) in shake flasks, supplemented with the deficient valine. The pH value of the synthetic medium was adjusted to 6.3 with potassium hydroxide. As a carbon source, xylose was autoclaved separately and added to the pre-culture medium at 2% (w/v) and to the fermentation medium at 4% (w/v), respectively. The fermentation medium also contained sodium formate 2.92 g/L, sodium acetate 4.1 g/L and sodium sulfate 14.2 g/L. See Table 16 for isobutanol yield of engineering Saccharomyces cerevisiae.

TABLE 16
Isobutanol yield of engineering Saccharomyces cerevisiae

| Chassis | | Yield of isobutanol |
|---|---|---|
| strain | | (g/L) |
| | Mutant with insertion of TRK1 | |
| CIBT7866 | Not integrated | 1.52 |
| | A1170T | 2.36 |
| | A1170V | 2.31 |
| | A1170M | 2.26 |
| | D1182Y | 2.21 |
| | P905H | 2.24 |
| | P905S | 226 |
| | L988S | 2.19 |
| | E764K | 2.18 |
| CIBT7866 | PUF2 gene mutant | |
| | Not mutated | 1.52 |
| | R243A | 2.49 |
| | R243C | 2.39 |
| | R243D | 2.36 |
| | R243E | 2.32 |
| | R243G | 2.18 |
| | R243L | 2.26 |
| | R243M | 2.29 |
| | R243N | 2.21 |
| | R243P | 2.24 |
| | R243S | 2.20 |
| | R243W | 2.18 |
| | puf2Δ (deletion) | 2.15 |
| | deletion of A at position 305 | 2.05 |
| | upstream of the PUF2 | |

As the comparison results in Table 16, when mutating A1170T, A1170V, A1170M, D1182Y, P905H, P905S, L988S, and E764K of TRK1 gene of the genome of the isobutanol-producing chassis strain CIBT7866 in its genome, the isobutanol production ability of the recombinant strains increased to varying degrees. When the genome of isobutanol-producing chassis strain CIBT7866 undergoes deletion mutations of R243A, R243C, R243D, R243E, R243G, R243L, R243M, R243N, R243P, R243S, R243W, sequence deletion, and 305 A upstream of PUF2 of PUF2 gene, the isobutanol production ability of the recombinant strains also increased to varying degrees, indicating that the above-mentioned mutations of TRK1 or PUF2 enhance the ability of Saccharomyces cerevisiae to produce isobutanol.

The above-mentioned experiments show that by directional modification of the PUF2 gene and TRK1 gene in the genome of Saccharomyces cerevisiae that can utilize xylose or arabinose by genetic engineering, a recombinant Saccharomyces cerevisiae that can efficiently utilize glucose, xylose and arabinose can be constructed. The resulting engineered strain can also be cultured and proliferated normally under high sodium salt concentration, and can be used to convert fermentable sugars in cellulase hydrolysate into various products, such as ethanol, lactic acid, butanedioic acid, farnesene or isobutanol.

It should also be noted that the listing and discussion of a previously published document in this specification should not be construed as an admission that the document is prior art or common general knowledge.

1. A method for improving the rate of xylose and arabinose utilization in Saccharomyces cerevisiae; or a method for improving the rate of glucose and arabinose utilization in Saccharomyces cerevisiae; or a method for improving the rate of glucose, xylose and arabinose utilization in Saccharomyces cerevisiae; or a method for improving sodium salt tolerance of Saccharomyces cerevisiae; or a method for improving fermentation of ethanol, lactic acid, butanedioic acid, farnesene or isobutanol; wherein the method comprises the following steps:
fermenting said Saccharomyces cerevisiae with the ability to convert xylose and/or arabinose into ethanol, lactic acid, butanedioic acid, farnesene or isobutanol,
and optionally recovering the fermentation product;
wherein said Saccharomyces cerevisiae comprises one or both of the following modifications in its genome:
A. a mutation in an endogenous TRK1 gene for the potassium ion intracellular transporter protein, resulting in an increase in intracellular potassium/sodium ratio in Saccharomyces cerevisiae, and/or
B. a mutation in an endogenous PUF2 gene for mRNA-binding protein or PUF2 gene knockout, resulting in PUF2 inactivation, deletion, reduced function, or down-regulated mRNA translation strength.

2. The method of claim 1, wherein the Saccharomyces cerevisiae is an alcohol-producing yeast.

3. The method of claim 1, wherein the mutation in the endogenous TRK1 gene in mode A is selected from mutations corresponding to positions 764, 905, 988, 1170 and/or 1182 of SEQ ID NO: 2.

4. The method of claim 3, wherein the mutation in the endogenous TRK1 gene in mode A is selected from the following mutations: D1182Y, A1170T, A1170M, A1170V, L988S, L988F, P905H, P905S, and E764K, or a combination of two or more thereof corresponding to SEQ ID NO: 2.

5. The method of claim 1, wherein the mutant encodes a variant having at least 80%, but less than 100%, sequence identity to the polypeptide of SEQ ID NO: 2.

6. The method of claim 1, wherein the mutated TRK1 gene coding sequence has at least 80%, but less than 100%, sequence identity to the TRK1 gene coding sequence of SEQ ID NO: 1.

7. The method of claim 1, wherein the endogenous TRK1 gene coding sequence being mutated is the TRK1 gene coding sequence of SEQ ID NO: 1.

8. The method of claim 1, wherein the mutation in the PUF2 gene in mode B is selected from one of the following mutations or from a combination of two or more of the following mutations:
B-1. mutation corresponding to position 243 of SEQ ID NO: 4 (e.g., R243);
B-2. PUF2 gene disruption;
B-3. deletion of A base at position 305 upstream of the PUF2 gene.

9. The method of claim 8, wherein the mutation in the endogenous PUF2 gene in mode B-1 is selected from the following mutations: R243A, R243C, R243D, R243E, R243G, R243L, R243M, R243N, R243P, R243S or R243W corresponding to SEQ ID NO: 4.

10. The method of claim 8, wherein the mutation in the endogenous PUF2 gene in mode B-1 encodes a variant having at least 80%, but less than 100%, sequence identity to the polypeptide of SEQ ID NO: 4.

11. The method of claim 8, wherein the mutated PUF2 gene coding sequence has at least 80%, but less than 100%, sequence identity to the PUF2 gene coding sequence of SEQ ID NO: 3.

12. The method of claim 8, wherein the endogenous PUF2 gene coding sequence being mutated is the PUF2 gene coding sequence of SEQ ID NO: 3.

13. The method of claim 1, wherein the chassis cell is a diploid Saccharomyces cerevisiae.

14. (canceled)

15. A recombinant Saccharomyces cerevisiae cell, wherein the cell has the ability to convert xylose and/or arabinose into ethanol, lactic acid, butanedioic acid, farnesene or isobutanol; and
wherein the cell has one or both of the following modifications in its genome:
A. a mutation in an endogenous TRK1 gene for the potassium ion intracellular transporter protein, resulting in an increase in intracellular potassium/sodium ratio in Saccharomyces cerevisiae, and/or
B. a mutation in an endogenous PUF2 gene for mRNA-binding protein or PUF2 gene knockout, resulting in PUF2 inactivation, deletion, reduced function, or down-regulated mRNA translation strength.

16. The recombinant Saccharomyces cerevisiae of claim 15, further comprising an exogenous xylose isomerase gene xylA and/or an arabinose metabolism-related gene expression cassette araBAD, and/or xylulose kinase and pentose phosphate pathway genes are enhanced, wherein the xylA is xylose isomerase gene XylA derived from Piromyces with a nucleotide sequence of SEQ ID NO: 1 disclosed in patent document CN 113736675 A, or xylose isomerase gene RuXylA derived from bovine rumen metagenome with a nucleotide sequence of SEQ ID NO: 2 disclosed in patent document CN 113736675 A; the arabinose metabolism-related gene expression cassette araBAD is arabinose metabolism gene expression cassette AUC disclosed in the patent document CN 110872596 A, including sequentially from upstream to downstream a gene arab expression element with a base sequence of SEQ ID NO: 2, a gene araA expression element with a base sequence of SEQ ID NO: 1, a gene araD expression element with a base sequence of SEQ ID NO: 3, a gene GAL2 expression element with a base sequence of SEQ ID NO: 4 and a gene STP2 expression element with a base sequence of SEQ ID NO: 5.

17. The recombinant Saccharomyces cerevisiae of claim 15, wherein an Idh gene from Lactobacillus acidophilus ATCC4356 is integrated into the genome.

18. The recombinant Saccharomyces cerevisiae of claim 15, wherein genes SDH1, SDH2, IDH1 and IDP1 are mutated and/or knocked out.

19. The recombinant Saccharomyces cerevisiae of claim 15, wherein a BFS gene is overexpressed, the mevalonate pathway of the Saccharomyces cerevisiae host strain is enhanced, and/or the sterol synthesis pathway is weakened/inhibited.

20-21. (canceled)
